(19) United States
(12) Patent Application Publication
(10) Pub. No.: US 2006/0047105 A1
(43) Pub. Date: Mar. 2, 2006

(54) Polymer-supported reagent for the preparation of disulfide-bridged peptides

(76) Inventors: Krzysztof Darlak, Fisherville, KY (US); George Barany, Falcon Heights, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/165,609

(22) Filed: Jun. 23, 2005

Related U.S. Application Data
(60) Provisional application No. 60/582,320, filed on Jun. 23, 2004.

(51) Int. Cl. C07K 1/02; C08L 89/00

(52) U.S. Cl. 530/333; 525/54.1

(57) ABSTRACT

A reagent for preparation of disulfide-bridged peptides is provided that comprises an oxidative functionality bound to a cross-linked ethoxylate acrylate resin polymer. The reagent has the formula: 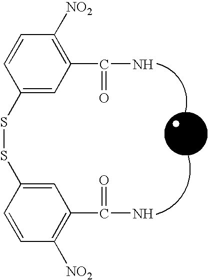 wherein \textcircled{R} is a cross-linked ethoxylate acrylate resin polymer. Methods of making and using this reagent are also described herein.

This application claims the benefit of U.S. Provisional Application No. 60/582,320, filed Jun. 23, 2004, which is hereby incorporated by reference.

This application is part of a government project. The research leading to this invention was supported from the Phase I SBIR Grant IR43 GM 58987 and in progress Phase II SBIR Grant 2R44 GM058987-O₂A1. The United States Government retains certain rights in this invention.

### FIELD OF INVENTION

The invention relates to formation of disulfide-bridged peptides from corresponding thiol precursors. More specifically, the present invention relates to polymer-supported reagents for formation of disulfide-bridged peptides from corresponding thiol precursors and methods of using same.

### BACKGROUND OF INVENTION

A number of disulfide-bridged peptides are of current and potential interest as therapeutic drugs, including oxytocin (childbirth), somatostatin and analogues (anticancer), vasopressin analogues (antidiuretic), calcitonin (osteoporosis), and integrelin (anticlotting). This may be due in part to the fact that pairing of cysteine residues to form disulfide bridges represents the principal way for Nature to establish covalent crosslinks that can “lock” conformations, with concomitant effects on structural stabilities and biological activities.

Approaches to form disulfides fall into three major classes: solution oxidation, oxidation of peptides while attached to a solid support, and use of polymer-bound oxidants. Commonly used oxidants, often in excess and each in appropriate aqueous and/or organic solutions, include potassium ferricyanide (K₃Fe(CN)₆), air, dimethyl sulfoxide (DMSO), glutathione redox buffers, iodine (12), or thallium trifluoroacetate (Tl(Tfa)₃). Some of the listed reagents are not fully compatible with the side-chains of sensitive amino acids such as tyrosine, methionine, and tryptophan, so side reactions can potentially occur. Also, some oxidation methods are quite sluggish, resulting in reaction times ranging from several hours to several days to effect completion. Even so, numerous problems can arise, including formation of dimers and oligomers, and pH-dependent solubility issues. Further limitations of the solution mode relate to the need to conduct reactions under high dilution reaction scales—this bears directly on scale-up; also some of the inorganic reagents used as oxidants are difficult to remove. The various methods may be ineffective or fail for challenging oxidations, and it is noteworthy that some of the more complex peptide targets reported on in the literature have been obtained in relatively low yields only after extensive optimization of experimental protocols for synthesis, purification, and oxidation/folding. Thus, despite the best efforts of peptide scientists worldwide, there remains a manifest need for improved, alternative approaches to disulfide formation that are convenient, robust, and reliable.

Polymer-supported reagents are increasing in popularity, since they combine the advantages of solid-phase chemistry with the versatility of solution-phase reactions. Thus, use of such reagents represents a way to achieve clean reactions, since excess materials, as well as contaminating by-products, can be removed easily by filtration. A polymer-bound oxidant for disulfide production was available commercially in the 1990's and sold as EKATHIOX™, but is no longer available. See PCT Publication No. WO 96/07676 by Brian R. Clark et al. for “Polymeric Resin For Disulfide Bond Synthesis,” published in March of 1996. In 1998, a novel polymer-supported oxidant was introduced defining conditions for its use to facilitate the formation of disulfide-bridged peptides under very mild conditions. See the article “Novel Solid-Phase Reagents for Facile Formation of Intramolecular Disulfide Bridges in Peptides under Mild Conditions,” Ioana Annis, Lin Chen, and George Barany, J. Am. Chem. Soc. 1998, 120, 7226-7238. The chemistry was based on Ellman's reagent, 5,5′-dithiobis(2-nitrobenzoic acid) (DTNB), which is used classically for the quantitative determination of free thiol content in physiological fluids. The solid-phase approach depended on a detailed understanding of the mechanism of the Ellman's reaction, and operationally involved bivalent, covalent attachment of Ellman's reagent to suitable polymer supports. Additionally, see U.S. Pat. No. 5,656,707 granted Aug. 12, 1997 to Kempe et al. for “Highly Cross-Linked Polymeric Supports”; U.S. Pat. No. 5,910,554 granted Jun. 8, 1999 to Kempe et al. and PCT Publication No. WO 97/00273 for “Highly Cross-Lined Polymeric Supports”; the article “CLEAR: A Novel Family of Highly Cross-Linked Polymeric Supports for Solid-Phase Peptide Synthesis,” Maria Kempe and George Barany, J. Am. Chem. Soc. 1996, 118, 7083-7097; and the article “Application of solid-phase Ellman's reagent for preparation of disulfide-paired isomers of α-conotoxin SI,” Balazs Hargittai, Ioana Annis, and George Barany, Lett. Pept. Sci. 7, 47-52, 2000.

Most polymeric resins for traditional solid-phase synthesis are based on polystyrene and have been optimized for peptide and organic synthesis applications. The hydrophobic nature of polystyrene, and its lack of swelling in polar solvents such as water and/or lower alcohols, has limited its use in biochemical applications where hydrophilic environments are desired. Because of this, alternative supports were introduced that were based on polyamides and carbohydrates. Further research focused on improvements in chemical and physical properties, and compatibility with aqueous systems. This led to the development of supports that were based on hydrophobic polystyrene but were modified further by adding hydrophilic polyethylene glycol spacers, as for example in PEG-PS™, TentaGel™, and ArgoGel™. PEGA (acryloylated poly(N,N-dimethacrylamide-co-bisacrylamido-polyethylene glycol-co-monoacrylamido-polyethylene glycol)) resins embody a similar theme but avoid a hydrophobic component. The various resins just discussed are all based on low cross-linked matrices that can lead to internal collapse, difficulties in filtration, and/or lack of suitability in flow-through systems. Ellman's reagent (5,5′-dithiobis(2-nitrobenzoic acid) or “DTNB”), has been attached to polyethylene glycol-polystyrene (PEG-PS™), controlled-pore glass (CPG), or modified Sephadex supports.

A need exists for better materials and techniques for formation of disulfide-bridged peptides from corresponding thiol precursors.

### SUMMARY OF THE INVENTION

The formation of disulfide bonds in synthetic peptides is one of the more challenging transformations to achieve in peptide chemistry, in view of the possible formation of oligomeric by-products and other side reactions, as well as occasional solubility problems in aqueous oxidizing media. The present invention provides a reagent for formation of disulfide bonds that combines a unique oxidative functionality with an equally unique polymer support.

More specifically, a reagent for preparation of disulfide-bridged peptides is provided that comprises an oxidative functionality bound to a cross-linked ethoxylate acrylate resin polymer. The reagent has the formula: 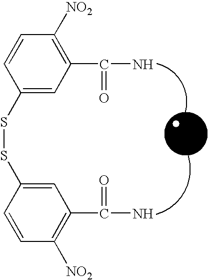
wherein \textcircled{R} is a cross-linked ethoxylate acrylate resin polymer prepared by reacting an olefin-containing monomer and a multifunctional (meth)acrylate crosslinker. The multifunctional (meth)acrylate crosslinker has the following formula: 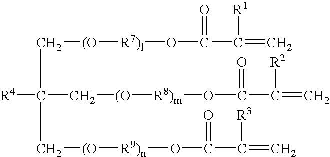
wherein:
(i) R¹, R², and R³ are each independently hydrogen or a methyl group,
(ii) R⁴ is hydrogen or an organic group or substituent that can interact in the polymerization and/or crosslinking process or is nonreactive under the conditions of the polymerization and/or crosslinking process,
(iii) R⁷, R⁸, and R⁹ are each independently —CH₂—CH₂—, —CH₂—CH₂—CH₂—, —CH₂—CH(CH₃)—, or —CH(CH₃)—CH₂—, and
(iv) each of l, m, and n is no greater than about 100 with the proviso that at least one of l, m, or n is at least 1. An embodiment of the polymer support as described herein is also referred to in this disclosure as CLEAR™ resin, and the reagent is also referred to in this disclosure as CLEAR-OX™ reagent.

Methods of making and using this reagent are also described herein.

This reagent surprisingly is capable of carrying out intramolecular thiol conversion to disulfide bonds with improved purities and yields, and improved ease of synthesis. The reagent of the present invention is an effective, reliable, and scalable reagent for converting the appropriate linear precursors into the corresponding intramolecular disulfides, and for isolating pure products by a straightforward procedure. This holds true even for structures that are difficult to oxidatively cyclize due to conformational issues. A particular advantage of the present reagent is the capability to work at a wide range of pH values, and to utilize conditions that are minimally deleterious towards labile sensitive side-chains. Furthermore, the present reagent is capable of carrying out oxidations with higher yields and purities at peptide concentrations at least 10-fold higher than the corresponding control oxidations carried out in solution. Additionally, the present reagent can be regenerated and recycled.

### BRIEF DESCRIPTION OF THE DRAWINGS

A better understanding of the present invention will be had upon reference to the following description in conjunction with the accompanying drawings in which like numerals refer to like parts throughout the several views and wherein:

FIG. 1. Proposed mechanism for CLEAR-OX™ mediated formation of intramolecular disulfide.

FIG. 2. Reaction sequence for preparation of the polymer-bound oxidant by formation of the final oxidant on the solid support.

FIG. 3. Reaction sequence for preparation of an anchored oxidant that can be directly or indirectly attached to the solid support.

FIG. 4. Reaction sequence for preparation of the polymer-bound oxidant by reaction of an anchored oxidant with a spacer modified solid support.

FIG. 5. Reaction sequence for preparation of S-xanthenyl protected Ellman's Reagent, S-Xan-TNB for use in reaction sequence of FIG. 2.

FIG. 6. HPLC comparison of (A) solution-phase oxidation at pH 7.5-8.0 vs. (B) CLEAR-OX™ mediated oxidation at pH 4.6 of crude peptide, H-Asp-c[Pen-Phe-Trp-Lys-Tyr-Cys]-Val-OH. HPLC conditions: Vydac C18, 218TP54, 5%-65% B in 50 min, 1 mL/min, wavelength 220 nm, A: 0.05% TFA in H₂O and B: 0.05% TFA in CH₃CN.

### DETAILED DESCRIPTION

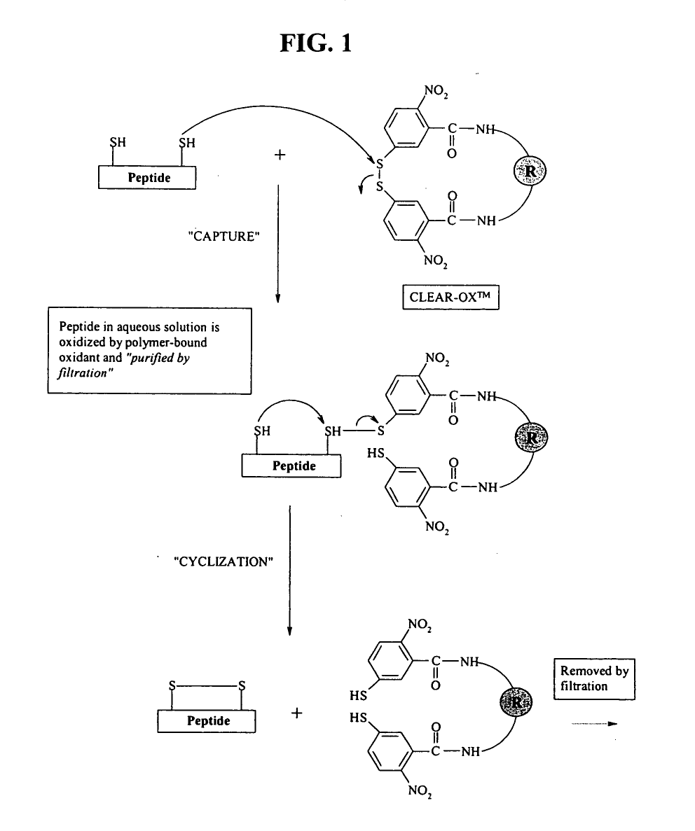

As noted above, a reagent is provided for formation of disulfide bonds that combines a unique oxidative functionality with an equally unique polymer support. The proposed mechanism for carrying out this disulfide bond formation is set forth in FIG. 1. As shown, an initial “capture” step is carried out, with reaction of one of the peptide-thiol groups with the solid phase reagent to provide a support-bound activated intermediate. Next, this intermediate undergoes intramolecular “cyclization” through attack by the other peptidyl thiol group, resulting in formation of the desired disulfide bridge and concomitant release of the monomeric oxidized peptide product back into solution. During the second step, the substrate is relatively sequestered (pseudodilution) from other potential thiol nucleophiles in solution or at other sites on the support, lessening the likelihood of competing intermolecular attacks which would lead to dimeric and oligomeric byproducts.

In one aspect of the present invention, a method is provided for preparing disulfide-bridged peptides comprising contacting a peptide solution comprising a peptide having two or more thiol functionalities (i.e. polythiol peptides) with the reagent as described herein under conditions suitable for oxidation of the thiol functionalities to form peptides having intramolecular peptide disulfide bonds. In another aspect of the present invention, the peptide solution of this method comprises two or more polythiol peptides as a peptide mixture, and the peptide solution is contacted with the reagent as described herein under conditions suitable for oxidation of the thiol functionalities to form a corresponding mixture of peptides having intramolecular peptide disulfide bonds.

These methods are particularly advantageous because the peptide solution concentration can be much higher than conventionally used in intramolecular disulfide bridge formation. Preferably, the peptide solution has peptide a concentration of from about 4 mg/ml to about 7 mg/ml. Additionally, it has surprisingly been found that the ratio of excess reagent to reduced peptide can be substantially lower than is conventionally used in intramolecular peptide disulfide bridge formation. Preferably, the ratio of excess reagent to reduced peptide is from about 2 to about 5.

Because of the unique solvent interaction characteristics of the present reagent, surprisingly advantageous solvent mixtures can be used. Preferably, the peptide solution comprises an organic solvent/aqueous mixed solvent system. In a particularly preferred embodiment, the peptide solution comprises an acetonitrile/aqueous mixed solvent system. Preferably this system comprises a buffer to control the pH of the media.

As noted above, the reagent of the present invention comprises an oxidative functionality bound to a cross-linked ethoxylate acrylate resin polymer. The oxidative functionality can be bound directly to the cross-linked ethoxylate acrylate resin polymer, or can be bound via a spacer moiety. The usage of a spacer moiety is useful to extend the functionality and improve accessibility by introducing a linker between the polymer and the polymer-supported reagent. While not being bound by theory, it is believed that a spacer moiety provides improved accessibility of the oxidant to the peptide thiol, resulting in faster reaction times and higher yields. The spacer moiety can be any appropriate connective functionality, such as a hydrocarbon linking group optionally interrupted by oxygen, sulfur or nitrogen atoms. Preferred such linking groups are alkylene linking groups or alkoxyalkyl linking groups. A particularly preferred spacer moiety is a linking group comprising one or more amino acid residues.

A particularly preferred reagent of the present invention has the formula: 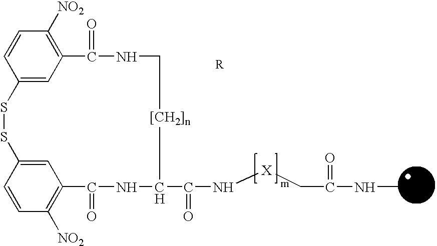
wherein n=1-8,
X=(CH₂) or (CH₂CH₂O)
and m=0-12.

Most particularly preferred reagents of this formula are reagents wherein n=4.

Another particularly preferred reagent of the present invention has the formula: 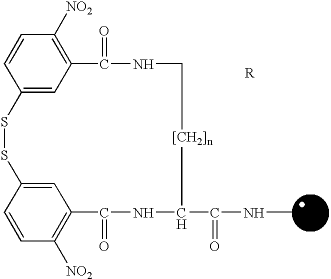
wherein n=1-8.

Most particularly preferred reagents of this formula are reagents wherein n=4.

The resin support portion of this reagent is specifically selected to be a cross-linked ethoxylate acrylate resin polymer. The resin portion alone of this reagent has been previously described in U.S. Pat. No. 5,656,707 issued on Aug. 12, 1997 to Maria Kempe and George Barany, and also in U.S. Pat. No. 5,910,554, the disclosures of which are incorporated by reference herein. The resin portion alone has been discussed in the literature, and identified as CLEAR™ (Cross-Linked Ethoxylate Acrylate Resin) polymeric supports. CLEAR™ polymeric supports per se are prepared using conventional technology as discussed herein and also in the above cited US patents.

The resin support used in the reagent of the present invention can be prepared from polymers having a wide range of molecular weights. The resin support can also have a wide range of pore sizes, porosities, surface areas, etc., depending on the desired end use. Although they can also be prepared with a wide range of crosslinking, they are preferably highly crosslinked (i.e., prepared using at least about 10 mole-% total crosslinker).

As noted above, the cross-linked ethoxylate acrylate resin polymer is prepared by reacting an olefin-containing monomer and a multifunctional (meth)acrylate crosslinker, wherein the multifunctional (meth)acrylate crosslinker has the following formula: 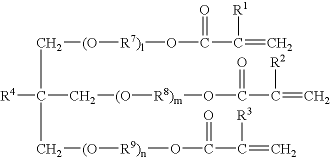
wherein:
(i) R¹, R², and R³ are each independently hydrogen or a methyl group,
(ii) R⁴ is hydrogen or an organic group or substituent that can interact in the polymerization and/or crosslinking process or is nonreactive under the conditions of the polymerization and/or crosslinking process,
(iii) R⁷, R⁸, and R⁹ are each independently —CH₂—CH₂—, —CH₂—CH₂—CH₂—, —CH₂—CH(CH₃)—, or —CH(CH₃)—CH₂—, and
(iv) each of l, m, and n is no greater than about 100 with the proviso that at least one of l, m, or n is at least 1.

Preferably, the sum of 1+m+n is from about 5 to about 25, and most preferably the sum of 1+m+n is about 14.

The CLEAR™ polymeric supports are prepared from multifunctional oxyacetylene- or oxypropylene-containing (meth)acrylate crosslinkers, and olefin-containing crosslinkers, preferably with an olefin-containing monomer (i.e., olefinic monomer), more preferably a functionalized olefin-containing monomers, and most preferably an amine-functionalized olefin-containing monomer.

The crosslinkers are polymerized with one or more olefinic monomers optionally functionalized with amino groups, carboxyl groups, hydroxyl groups, and the like. The synthesis of the resin support used in the reagent of the present invention is advantageous because it can occur in one step.

As used herein, an organic group or substituent is nonreactive under the conditions of the polymerization and/or crosslinking process if it does not undergo chemical change or transformation during the reaction and does not prevent the reaction. By this it is meant that the nonreactive group is selected such that the intended reactive components that form the support resin can react in the manner described. An organic group or substituent interacts in the polymerization and/or crosslinking process if it reacts with the olefinic monomer or other crosslinker molecules to cause chain growth or crosslinking. Suitable R⁴ groups include substituents such as hydroxyl groups, carboxyl groups, amide groups, ester groups, halogens, amine groups, and the like, as well as alkyl groups, aryl groups, alkaryl or aralkyl groups, alkenyl groups, alkynyl groups, and the like, which can optionally include nonperoxidic oxygen, sulfur, or nitrogen atoms, and be unsubstituted or substituted with the substituents listed above. Preferably, R⁴ is hydrogen, an oxyacetylene-containing or oxypropylene-containing (meth)acrylate group, an alkyl group, or a hydroxyalkyl group. More preferably, R⁴ is hydrogen, —CH₂—(O—CH₂—CH₂)ₓO—C(O)—C(R⁵)═CH₂ wherein R⁵ is hydrogen or methyl group and x is no greater than about 100 (preferably 1-30), a (C₁-C₄)alkyl group, or a hydroxy(C₁-C₄)alkyl group. Thus, the multifunctional oxyacetylene- or oxypropylene-containing (meth)acrylate crosslinkers can be tri- or tetra-functional acrylates or methacrylates.

The support resin preferably is based on the key cross-linking component trimethylolpropane ethoxylate (14/3 EO/OH) triacrylate. This building block contains relatively short chains (four to five ethylene oxide (EO) units), in contrast to other PEG-containing resins (typically 20-70 EO units), yet has no aromatic component such as polystyrene. The short EO chains are distributed uniformly throughout the very highly cross-linked, polymer matrix. The unique branched structure gives this support excellent swelling properties in a broad spectrum of solvents such as tetrahydrofuran (THF), dichloromethane (CH₂Cl₂), and N,N-dimethylformamide (DMF), as well as water and alcohols.

In a particularly preferred embodiment, the multifunctional (meth)acrylate crosslinker has the formula:

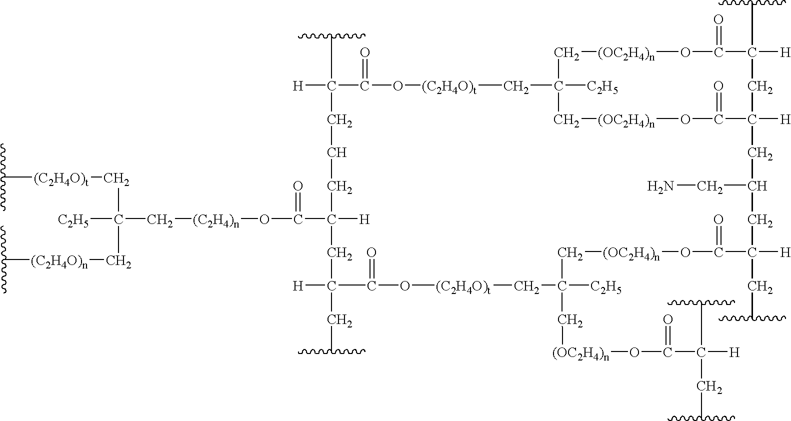

Typically, the CLEAR™ polymers are prepared using a high level of crosslinker (i.e., at least about 10 mole-%, based on the total number of moles of reactants). Preferably, the polymers of the present invention are prepared using at least about 15 mole-% total crosslinker, more preferably at least about 25 mole-%, and most preferably at least about 50 mole-% total crosslinker. The total amount of crosslinker can be as high as 98 mole-% and even up to 100 mole-%, and still produce a polymer with good swelling properties. The total amount of crosslinker includes the multifunctional oxyacetylene- or oxypropylene-containing (meth)acrylate crosslinkers and any optional secondary olefin-containing crosslinkers.

The secondary olefin-containing crosslinkers include any crosslinkers typically used in crosslinking polymers made from olefinic and/or (meth)acrylate monomers. Generally, such crosslinkers are of the formula H₂C═CH—R⁶—HC═CH₂ or H₂C═C(CH₃)—R⁶—(H₃C)C═CH₂, wherein R⁶ is a divalent organic group, which may be linear, cyclic, or branched containing aromatic and/or aliphatic moieties and optional functionalities such as amide groups, carboxyl groups, nonperoxidic oxygen atoms, and the like. Examples of such secondary crosslinkers include, but are not limited to, divinylbenzene, ethylene glycol dimethacrylate [H₂C═C(CH₃)—C(O)—O—CH₂—CH₂—O—C(O)—(CH₃)C═CH₂], poly(ethylene glycol-400)-dimethacrylate [H₂C═C(CH₃)—C(O)—(O—CH₂—CH₂)₉—O—C(O)—(CH₃)C═CH₂], N,N′-methylenediacrylamide [H₂C═CH—C(O)—NH—CH₂—NH—C(O)—CH═CH₂], N,N′-1,4-phenylenediacrylamide [H₂C═CH—C(O)—NH—C₆H₄—NH—C(O)—CH═CH₂], 3,5-bis(acryloylamido)benzoic acid [H₂C═CH—C(O)—NH—C₆H₃(CO₂H)—NH—C(O)—CH═CH₂], and N,O-bisacryloyl-L-phenylalaninol [H₂C═CH—C(O)—NH—CH(CH₂—C₆H₅)—CH₂—O—C(O)—CH═CH₂]. The secondary olefin-containing crosslinker may also be multi-functional (meth)acrylate crosslinkers as in formula I wherein l, m, and n are each 0, such as pentaerythritol triacrylate (wherein l, m, and n each are 0, R¹, R², and R³ are each H, and R⁴ is an OH group), trimethylolpropane trimethacrylate (wherein l, m, and n each are 0, R¹, R², and R³ are each CH₃, and R⁴ is —CH₂ CH₃ group), and pentaeryiritol tetraacrylate (wherein l, m, and n each are 0, R¹, R², and R³ are each H, and R⁴ is —CH₂—O—C(O)—CH═CH₂). Preferably, the secondary olefin-containing crosslinker is selected from the group consisting of a diacrylate, a dimethacrylate, a diacrylamide, a dimethacrylamide, and a divinylbenzene.

The crosslinkers are copolymerized with one or more olefinic monomers optionally functionalized with amino groups, carboxyl groups, hydroxyl groups, etc. Generally, the functional groups serve as starting points for substituents that will be coupled to the polymeric support. These functional groups can be reactive with an organic group that is to be attached to the solid support or it can be modified to be reactive with that group, as through the use of linkers or handles. The functional groups can also impart various desired properties to the polymer, depending on the use of the polymers. For example, if used in ion exchange chromatography, the polymers of the present invention should include charged groups. If used as supports for peptide synthesis, the polymers of the present invention can include amino groups. Preferably, the polymers of the present invention are made using olefinic monomers containing amino functional groups.

Suitable olefins (i.e., olefinic monomers) include, for example, vinyl carboxylic acids such as acrylic acid, methacrylic acid, itaconic acid, and vinylbenzoic acid; vinyl esters such as vinyl acetate, vinyl propionate, and vinyl pivalate; allyl esters such as allyl acetate; allyl amines such as allyl amine and allylethylamine; acrylic esters such as methyl acrylate, cyclohexylacrylate, benzylacrylate, isobornyl acrylate, hydroxybutyl acrylate, glycidyl acrylate, and 2-aminoethyl acrylate; methacrylic esters such as methyl methacrylate, butyl methacrylate, cyclohexyl methacrylate, benzyl methacrylate, ethyl methacrylate, glycidyl methacrylate, and 2-aminoethyl methacrylate; vinyl acid halides such as acryloyl chloride and methacryloyl chloride; styrene and substituted styrenes such as 4-ethylstyrene, 4-aminostyrene, dichlorostyrene, chlorostyrene, 4-hydroxystyrene, hydroxymethylstyrene, 4-hydroxy-3-nitro-styrene, 3-hydroxy-4-metoxy-styrene, and vinylbenzyl alcohol; vinyltoluene; heteroaromatic vinyls such as 1-vinylimidazole, 4-vinylpyridine, and 2-vinylpyridine; mono-functional oxyacetylene-containing (meth)acrylates such as poly(ethylene glycol) ethyl ether methacrylate [H₂C═C(CH₃)—C(O)—O—(CH₂—CH₂—O)_{q}—CH₂—CH₃ wherein q=3-5]; hydroxyl-containing (meth)acrylates such as 3-chloro-2-hydroxypropyl (meth)acrylate and hydroxyalkyl (meth)acrylates wherein the alkyl moiety contains 2-7 carbon atoms (e.g., 2-hydroxyethyl (meth)acrylate, 3-hydroxypropyl (meth)acrylate, 4-hydroxybutyl (meth)acrylate, 2-hydroxybutyl (meth)acrylate, 2-hydroxypropyl (meth)acrylate, 5-hydroxypentyl (meth)acrylate, and 2,3-dihydroxypropyl (meth)acrylate; hydroxyl-containing caprolactone (meth)acrylates such as the ring opening addition products of s-caprolactone with 2-hydroxyethyl (meth)acrylate or 2-hydroxypropyl (meth)acrylate; poly(alkylene glycol)(meth)acrylates such as the ring opening addition products of ethylene oxide and/or propylene oxide with (meth)acrylic acid such as diethylene glycol (meth)acrylate, triethylene glycol (meth)acrylate, and polyethylene glycol methacrylate, and polypropylene glycol methacrylate; hydroxyl-containing (meth)acrylamides such as N-(hydroxymethyl)(meth)acrylamide, N-(1-hydroxyethyl)(meth)acrylamide, N-(2-hydroxyethyl)(meth)acrylamide, N-methyl-N-(2-hydroxyethyl)(meth)acrylamide, N-(1-hexyl-2-hydroxy-1-methylethyl) (meth)acrylamide, N-propyl-N-(2-hydroxyethyl(meth)acrylamide, N-cyclohexyl-N-(2-hydroxypropyl)(meth)acrylamide, -bromo-N-(hydroxymethyl) acrylamide, and -chloro-N-(hydroxymethyl)acrylamide); allyl alcohols such as allyl alcohol, 1-buten-3-ol, 1-penten-3-ol, 1-hexen-3-ol, 1-hydroxy-1-vinyl cyclohexane, 2-bromoallyl alcohol, 2-chloroallyl alcohol, 2-methyl-1-buten-3-ol, 2-ethyl-1-penten-3-ol, and 1-phenyl-2-propen-1-ol; hydroxyl-containing vinyl ethers such as hydroxyethyl vinyl ether and hydroxybutyl vinyl ethers); and hydroxyl-containing allyl ethers such as allyl-1-methyl-2-hydroxyethyl ether, allyl-2-hydroxypropyl ether, allyl-2-hydroxy-1-phenyl ether, and allyl-2-hydroxy-2-phenyl ether. It should be understood that one or more types of olefinic monomers can be used to make the polymer supports. Depending on the end use, one can choose the desired combination of monomers and the desired type and amount of functionalization.

The polymer supports can be made using optional ingredients such as free-radical initiators (e.g., thermolytic and/or photolytic initiators). Such free-radical initiators include those normally suitable for free-radical polymerization of acrylate monomers. These species include azo compounds, tertiary amines, as well as organic peroxides, such as benzoyl peroxide and lauryl peroxide, and other initiators. Examples of azo compounds include 2,2′-azobis(2-methylbutyronitrile) and 2,2′-azobis(isobutyronitrile). Commercial products of this type include VAZO 67, VAZO 64 and VAZO 52 initiators supplied by E.I. duPont de Nemours & Co. Typically about 0.1-2.0 wt-% is used based upon the total monomer weight.

The unique swelling properties of this highly crosslinked support in both organic and aqueous solvents makes this polymer-supported oxidant superior in formation of disulfide bonds, and especially in the case of difficult to solubilize peptides.

TABLE I
CLEAR RESIN SUPPORTS
Swelling Properties of CLEAR Polymetric Support

| | Bed Volume (ml) |
|---|---|
| Solvent | of 1 g of resin |
| CH₂Cl₂ | 7.5 |
| DMF | 7.0 |
| MeCN | 7.0 |
| THF | 6.0 |
| MeOH | 6.0 |
| H₂O | 5.5 |

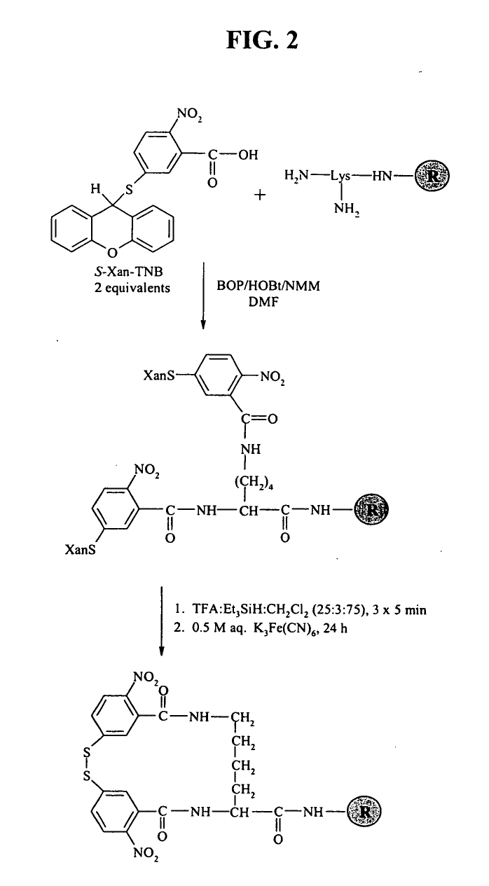

The polymer-supported oxidant, CLEAR-OX™, can be prepared by at least two synthetic routes. In the first approach as shown in FIG. 2, formation of the final oxidant (Ellman's reagent) is conducted on the solid support (CLEAR™). This is achieved via attachment of a sulfur protected 2-nitro-5-thiobenzoic acid (TNB) (using xanthenyl or other protecting group) to the resin-bound bifunctional amino acid anchor (preferably lysine) with a spacer moiety (Spacer X=(CH₂)ₘ or (CH₂CH₂O)ₘ where m=0-12) or without a spacer moiety between the polymer backbone and the bifunctional anchor. The sulfur protecting group is then removed and the thiol functionalities are subsequently oxidized to form 5,5′-dithiobis(2-nitrobenzoic acid) (DTNB Ellman's reagent) bound to the solid support (thereby forming CLEAR-OX™ reagent.

More specifically, a method of making the reagent as described herein is provided comprising:
a) providing a cross-linked ethoxylate acrylate resin polymer by reacting an olefin-containing monomer and a multifunctional (meth)acrylate crosslinker, wherein the multifunctional (meth)acrylate crosslinker has the following formula: 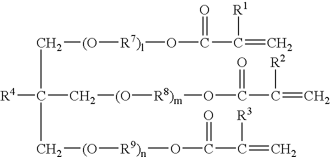
   wherein:
(i) R¹, R², and R³ are each independently hydrogen or a methyl group,
(ii) R⁴ is hydrogen or an organic group or substituent that can interact in the polymerization and/or crosslinking process or is nonreactive under the conditions of the polymerization and/or crosslinking process,
(iii) R⁷, R⁸, and R⁹ are each independently —CH₂—CH₂—, —CH₂—CH₂—CH₂—, —CH₂—CH(CH₃)—, or —CH(CH₃)—CH₂—, and
(iv) each of l, m, and n is no greater than about 100 with the proviso that at least one of l, m, or n is at least 1;
b) binding a bifunctional amino acid anchor to the cross-linked ethoxylate acrylate resin polymer;
c) attaching two 2-nitro-5-thiobenzoic acid compounds wherein the sulfur is protected by a sulfur protecting group to the resin-bound bifunctional amino acid anchor to form two sulfur protected 2-nitro-5-thiobenzoic acid residues;
d) removing the sulfur protecting groups; and
e) oxidizing the two 2-nitro-5-thiobenzoic acid residues to form 5,5′-dithiobis(2-nitrobenzoic acid residues bound to the to the cross-linked ethoxylate acrylate resin polymer.

Preferably, the bifunctional amino acid anchor comprises a lysine residue.

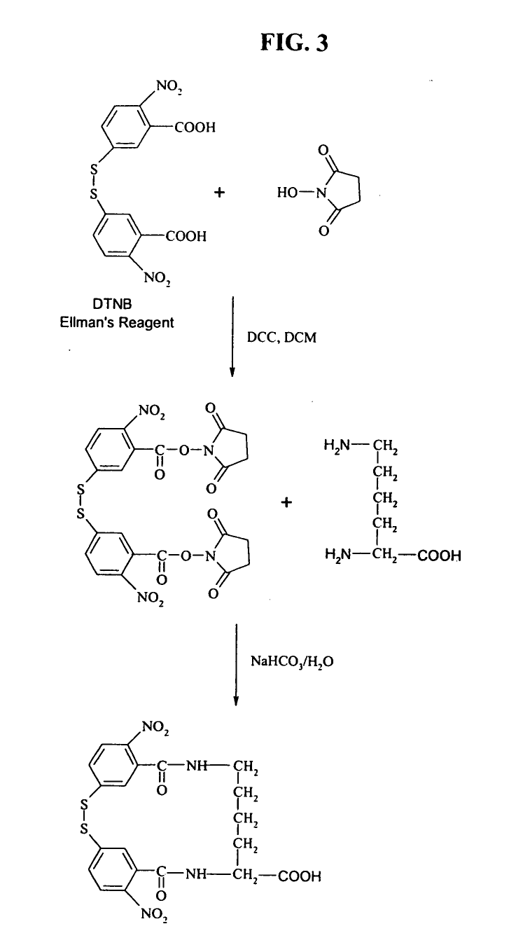

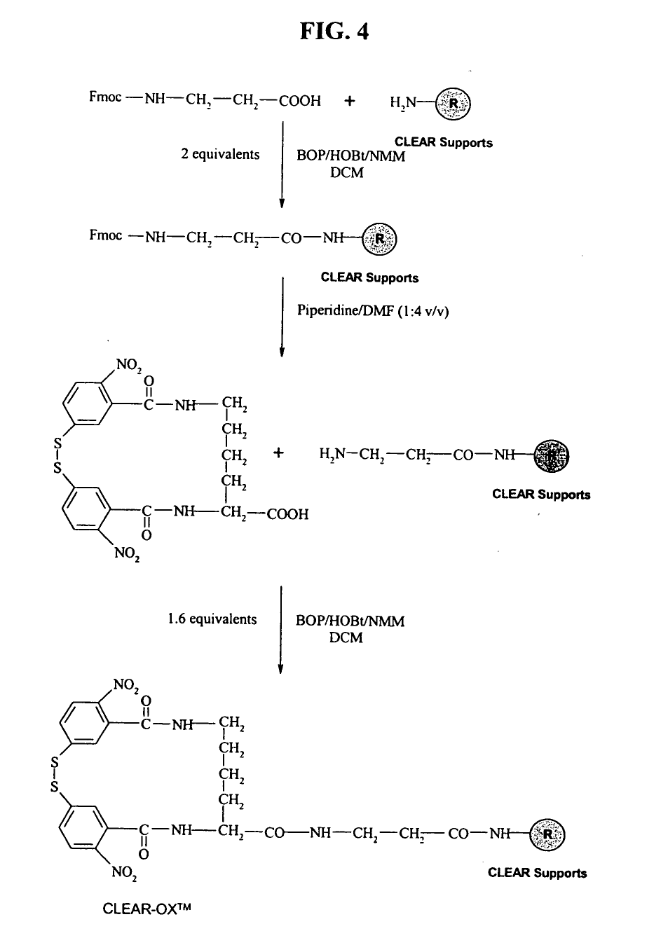

In a second synthetic route of CLEAR-OX™, a bifunctional anchor (preferably lysine) is reacted in solution with a preactivated Ellman's reagent [5,5′-dithiobis(2-nitrobenzoic acid)] to form the final DTNB-lysine derivative as shown in FIG. 3. This final DTNB-lysine derivative is bound either directly to the CLEAR™ polymeric support or to the spacer modified CLEAR™ polymeric support as shown in FIG. 4 to yield the final CLEAR-OX™.

More specifically, a method of making the reagent as described herein is provided, comprising:
a) providing a cross-linked ethoxylate acrylate resin polymer by reacting an olefin-containing monomer and a multifunctional (meth)acrylate crosslinker, wherein the multifunctional (meth)acrylate crosslinker has the following formula: 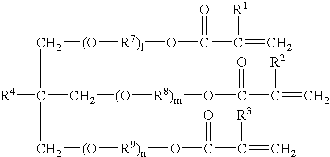
   wherein:
(i) R¹, R², and R³ are each independently hydrogen or a methyl group,
(ii) R⁴ is hydrogen or an organic group or substituent that can interact in the polymerization and/or crosslinking process or is nonreactive under the conditions of the polymerization and/or crosslinking process,
(iii) R⁷, R⁸, and R⁹ are each independently —CH₂—CH₂—, —CH₂—CH₂—CH₂—, —CH₂—CH(CH₃)—, or —CH(CH₃)—CH₂—, and
(iv) each of l, m, and n is no greater than about 100 with the proviso that at least one of l, m, or n is at least 1;
b) binding a bifunctional amino acid anchor in solution with 5,5′-dithiobis(2-nitrobenzoic acid) (“DTNB”) to form a DTNB-bifunctional amino acid anchor derivative; and
c) binding the DTNB-bifunctional amino acid anchor derivative to the cross-linked ethoxylate acrylate resin polymer.

Preferably, the bifunctional amino acid anchor comprises a lysine residue. In one embodiment of this method, the DTNB-bifunctional amino acid anchor derivative is bound directly to the cross-linked ethoxylate acrylate resin polymer. In another embodiment of this method, the DTNB-bifunctional amino acid anchor derivative is bound to the cross-linked ethoxylate acrylate resin polymer via a spacer moiety.

The reagent so prepared may be provided in any form suitable for use in carrying out the formation of disulfide bridges as described herein. Preferably, the reagent is provided in the form of beads or particles.

The following examples describe preferred embodiments of the invention. Other embodiments within the scope of the claims herein will be apparent to one skilled in the art from consideration of the specification or practice of the invention as disclosed herein.

### EXAMPLES

Amino acids and peptides are abbreviated and designated following the rules of the IUPAC-IUB Commission of Biochemical Nomenclature. Amino acid symbols denote the L-configuration unless noted otherwise. The following additional abbreviations are used: Ac₂O, acetic anhydride; AcOH, acetic acid; BOP, (benzotriazol-1-yl-N-oxy)tris(dimethylamino)phosphonium hexafluorophosphate; CLEAR™, Cross-Linked Ethoxylate Acrylate Resin; CLEAR-OX™, Cross-Linked Ethoxylate Acrylate Resin-bound Oxidant; CPG, controlled-pore glass; DMF, N,N-dimethylformamide; DMSO, dimethyl sulfoxide; DTNB, 5,5′-dithiobis(2-nitrobenzoic acid) (Ellman's reagent); EO, ethylene oxide; EtOAc, ethyl acetate; ESI-TOF, electrospray ionization-time of flight (mass spectrometry); ES-MS, electrospray mass spectrometry; Et₃N, triethylamine; HBTU, 2-(1H-benzotriazol-1-yl)-1,1,3,3-tetramethyluronium hexafluorophosphate; HOBt, 1-hydroxybenzotriazole; MeOH, methanol; NMM, N-methylmorpholine; PEG, polyethylene glycol; PEG-PS, polyethylene glycol-polystyrene graft resin support; THF, tetrahydrofuran; Tl(Tfa)₃, thallium trifluoroacetate; TIPS, triisopropylsilane; TFA, trifluoroacetic acid; TNB, 2-nitro-5-thiobenzoic acid; U II, urotensin II; Xan, 9H-xanthen-9-yl.

Analytical grade solvents (“Baker Analytical”) were purchased from Mallinckrodt Baker (Phillipsburg, N.J.). Ellman's reagent [5,5′-dithiobis(2-nitrobenzoic acid), (DTNB)], 9H-xanthen-9-ol, N-methylmorpholine (NMM), and piperidine were purchased from Aldrich Chemical (Milwaukee, Wis.). Fmoc-protected amino acids, coupling agents, and resins were obtained from Peptides International (Louisville, Ky.).

Peptide products were hydrolyzed in 6 N HCl (18-24 h, 110° C.), following which amino acid analysis was performed on a Shimadzu 10A HPLC system with fluorescence detection using the Accutag Method. No special precautions were taken to avoid degradation; therefore Cys and Trp values were not determined. Synthetic peptides were characterized by electrospray ionization-time of flight mass spectrometry (ESI-TOF) performed on a Mariner instrument (PE Applied Biosystem, Foster City, Calif.).

Thin-layer chromatography (TLC) was performed on Silica Gel 60 F₂₅₄ (Merck, Darmstadt, Germany), developed in the solvent system indicated for each case. Spots were visualized by (a) UV, (b) I₂ vapor, and/or (c) spraying with ceric-molybdate reagent followed by heating. Analytical HPLC was performed using Vydac C₁₈ columns (4.6×250 mm, 218TP54) on an Agilent 1100 system using gradients (1% per min) of 0.05% TFA in CH₃CN and 0.05% aqueous TFA, with detection at 220 nm. Preparative HPLC was performed on a Vydac C₁₈ column (10-15 μm particle size, 5×30 cm) on a Shimadzu 8A HPLC system. Peptides were eluted using a linear gradient of 0.05% TFA in CH₃CN and 0.05% aqueous TFA (0.5%/min), at 100 mL/min flow rate, with detection at 226 nm.

Peptide synthesis was carried out with a PE Biosystems Pioneer™ or a Milligen 9050 peptide synthesizer using standard, double-coupling cycles of Fmoc/tBu protocols with either benzotriazol-1-yl-N-oxy)tris(dimethylamino)phosphonium hexafluorophosphate (BOP) or 2-(1H-benzotriazol-1-yl)-1,1,3,3-tetramethyuronium hexafluorophosphate (HBTU) coupling reagents, in the presence of 1-hydroxybenzotriazole (HOBt) plus NMM in DMF. Side-chains of the amino acids used in the synthesis were protected as follows: Asn(Trt), Asp(OtBu), Arg(Pbf), Cys(Trt), Glu(OtBu), Gln(Trt), Pen(Trt), Om(Boc), Thr(tBu), Trp(Boc), and Tyr(tBu). Test sequences were assembled on Wang-Polystyrene, CLEAR Amide, or CLEAR Acid resins obtained from Peptides International. Cleavages of peptides, and concomitant final deprotections, were carried out with a TFA:phenol:H₂O:triisopropylsilane (TIPS) (88:5:5:2) cocktail mixture (10 mL per g peptide-resin; argon was bubbled through the cocktail for 5 min prior to addition to resin) for 2 h at 25° C. under an argon blanket. The resins were filtered and washed with a small amount of cleavage cocktail. Combined filtrates were evaporated under reduced pressure, the residual product was precipitated with Et₂O:TIPS (99:1) and the peptide was collected by filtration and then dried in vacuo. The crude peptides so obtained were determined by analytical HPLC to have initial purities ranging from 40-80%, but they were used directly, without further purification, in experiments to form the disulfide either in solution or as mediated by CLEAR-OX™.

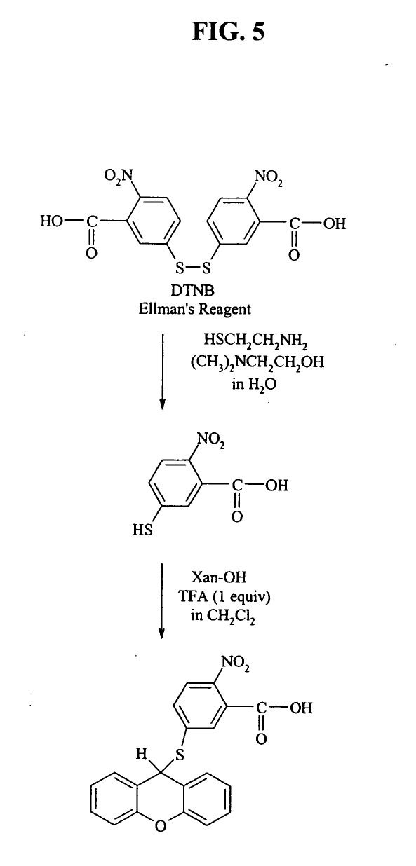

Commercially available 5,5′-dithiobis(2-nitrobenzoic acid) (Ellman's reagent) was transformed further as shown in FIG. 5, following Annis et al. Alternatively other reagents such as NaBH₄, or phosphines can be used to produce TNB. The key S-xanthenyl-protected 2-nitro-5-mercaptobenzoic acid derivative (S-Xan-TNB) was obtained on a 20 gram scale in an overall yield of 81% based on DTNB. Subsequently, (i) CLEAR™ support was converted to Fmoc-Lys(Fmoc)-CLEAR™; (ii) Fmoc groups were removed; (iii) S-Xan-TNB was attached to both pendant (N^{α} and N^{ε}) amines; (iv) S-protection was removed with acid; and (v) intraresin aromatic disulfide formation was mediated by K₃Fe(CN)₆, reproducibly on scales ranging from 20 to 60 grams of resin as illustrated in FIG. 2.

### Xanthenyl-protected Ellman's reagent 2-nitro-5-S-(9H-xanthene-9-yl)thiobenzoic acid [S-Xan-TNB]

Xanthenyl-protected Ellman's reagent for use in the reaction scheme as shown in FIG. 2 was prepared on a 20 g scale from commercially available 5,5′-dithiobis(2-nitrobenzoic acid) (DTNB) by closely following the procedure of Annis et al. The reaction scheme for preparing the protected Ellman's reagent is shown in FIG. 5. Reduction of DTNB with β-mercaptoethylamine in the presence of N,N-dimethyl-N-(2-hydroxyethyl)amine gave 2-nitro-5-thiobenzoic acid (TNB) in essentially quantitative yield. This material was reacted directly with 9H-xanthen-9-ol to provide the title product S-Xan-TNB in 70-78% yield, after crystallization from CH₂Cl₂:MeOH with addition of hexane, mp 174-176° C. dec, (literature 172°, Annis et al). TLC, single spot (CHCl₃:MeOH:AcOH (85:10:5 v/v/v)) R_{f} 0.77; (CHCl₃:MeOH:AcOH (90:8:2 v/v/v)) R_{f} 0.67; (EtOAc:Hexane:AcOH (1:1:0.01 v/v/v)) R_{f} 0.44. Elemental Analysis: theory C, 63.32; H, 3.45; N, 3.69; S, 8.45; found: C, 62.64; H, 3.58; N, 3.79; S, 8.53. ES-MS calc C₂₀H₁₃NO₅S: 379.05 found (negative mode m/z) 378.6 (M−H)⁻.

### CLEAR-OX™ Resin

CLEAR™ base HCl (20 g) (0.5 mmol/g) was pre-swollen in 300 mL CH₂Cl₂ for 12 h before use. All wash volumes were 150 mL, with wash times of 1 min, unless noted otherwise. The starting resin was washed with the following: CH₂Cl₂ (3×), Et₃N:CH₂Cl₂ (1:9 v/v, 2×2 min) to neutralize the HCl salt, CH₂Cl₂ (3×), and DMF (3×). Next Fmoc-Lys(Fmoc)-OH (11.8 g, 20 mmol), BOP (8.84 g, 20 mmol), and HOBt (3.06 g, 20 mmol) were combined and dissolved in 100 mL of DMF, and then NMM (3.7 mL, 34 mmol) was added. The combined solution was added to the resin, and shaking proceeded for 12 h. The resin was then washed with DMF (6×) and capped with 1 M Ac₂O and 1 M Et₃N in 150 mL of DMF for 45 min at 25° C., followed by washing with DMF (4×) and CH₂Cl₂ (3×).

A small portion of the resin was subjected to analysis for Fmoc group content, following the procedure of Grandas et al. (Anchoring of Fmoc-amino acids to hydroxymethyl resins, Int. J. Pept. Protein Res. 33, 386-390 (1989)): this step indicated a substitution level of 0.2 mmol/g. The bulk resin was washed with DMF (3×) and treated with piperidine:DMF (1:4 v/v, 2 min+10 min) to achieve Fmoc group removal. After washing with DMF (8×), the resin was reacted with S-Xan-TNB (4.56 g, 12 mmol), BOP (5.3 g, 12 mmol), HOBt (1.83 g, 12 mmol), and NMM (2.35 mL, 21.6 mmol) in DMF for 12 h, using the general coupling procedure already described above. After washing with DMF (6×) and CH₂Cl₂ (3×), completion of acylation was confirmed by a ninhydrin test.

Removal of the S-xanthenyl group was accomplished by treatment with TFA:CH₂Cl₂:TIPS (25:75:3 v/v/v, 3×5 min each). The resin was then washed with CH₂Cl₂ (6×), DMF (6×), and oxidized with a solution of K₃Fe(CN)₆ (16.45 g, 50 mmol) in 100 mL of DMF:H₂O (1:1, v/v), for 20 h at 25° C. Finally, the resin was washed extensively with the following: H₂O (6×), DMF (3×), H₂O (3×), DMF (3×), MeOH (3 x), CH₂Cl₂ (3×), and Et₂O (3×), and then dried in vacuo yielding 21.74 grams.

### Preparation of 5,5′ dithiobis(2-nitrobenzoic acid) N-hydroxysuccinimide Ester

N-hydroxysuccinimide (15.63 g, 136 mmol) and 5-5′ dithiobis(2-nitrobenzoic acid) (25 g, 63 mmol) were added to a 3 L 3-neck flask. The reagents were dissolved in 125 mL of DMF and diluted with 1000 mL of CH₂Cl₂. The flask was equipped with a mechanical stirrer, capped with a drying tube and cooled to 0° C. in an ice bath. DCC (28.25 g, 136 mmol) was dissolved in 250 mL of CH₂Cl₂ and added dropwise with stirring. Reaction progress was monitored by TLC EtOAc:MeOH:H₂O (5:1:0.75). Once the reaction was complete, the urea was removed via vacuum filtration, and the solution was concentrated to remove CH₂Cl₂. The concentrate was diluted with 500 mL of EtOAc and gravity filtered to remove any urea. The solution was again concentrated to remove EtOAc. The resulting concentrate was used for the next step.

### Preparation of N^{α},N^{ε}-Bis(5-thio-2-nitrobenzoyl)-L-lysine disulfide (DTNB-Lys-OH)

In a 12 L 3-neck flask equipped with a mechanical stirrer, a 5% aqueous NaHCO₃ solution was prepared by dissolving 200 g of NaHCO₃ in 4000 mL of water. L-Lys-OH×HCl (11.5 g, 63 mmol) was dissolved in the sodium bicarbonate solution with vigorous stirring. The 5,5′ dithiobis(2-nitrobenzoic acid) N-hydroxysuccinimide ester concentrate from the previous step was dissolved in 3750 mL of dioxane and added dropwise over 4-5 hours. The solution turned cloudy and got progressively more orange as the reaction proceeded. The solution was stirred overnight. The mixture was concentrated to remove dioxane and acidified to pH 2-3 using 6 N hydrochloric acid and vigorous stirring. The resulting cream colored solid was collected via vacuum filtration, washed 3 times with water, and dried overnight under high vacuum, yield=29.5 g. The crude material was recrystallized from 1600 mL of hot n-butanol/water (3:1) followed by 5 washes with H₂O, dried in vacuo, mp 228.4° C. dec, [α]_{D}²⁵=+187.3°. The product was found to be 95% pure by HPLC according to the following elution conditions: buffer A, 0.05% TFA in water and buffer B, 0.05% TFA in acetonitrile using a linear gradient of 20-60% buffer B over 40 min at a flow rate of 1 ml/min with detection at 220 nm. Calculated mass for C₂₀H₁₈N₄O₈S₂ was 506.06, and ES-MS positive mode found [M+H]⁺ of 507.07. TLC (EtOAc:MeOH:H₂O (5:1:0.75, v/v/v)) R_{f} 0.39; (CHCl₃:MeOH:H₂O:AcOH (60:18:3:3, v/v/v/v)) R_{f} 0.51. Elemental analysis: theory C, 47.43; H, 3.58; N, 11.06; S, 12.66; found: C, 47.24; H, 3.68; N, 10.77; S, 12.21.

### Preparation of CLEAR-OX™ Resin with β-Ala Linker

The CLEAR™ base×HCl (20 g) (0.5 mmol/g) was pre-swollen in 300 mL CH₂Cl₂ for 12 h before use. All wash volumes were 150 mL with wash times of 1 min, unless noted otherwise. The starting resin was washed with the following: CH₂Cl₂ (3×), (Et₃N):CH₂Cl₂ (1:9 (v/v), 2×2 min) to neutralize the HCl salt, CH₂Cl₂ (3×), and DMF (3×). Next, Fmoc-β-Ala-OH (6.23 g, 20 mmol) and HOBt (3.06 g, 20 mmol) were combined and dissolved in 100 mL DMF. The solution was added to the resin and shaken for 5 min. DICD (3.10 ml, 20 mmol) was added and shaking proceeded for 12 h. The resin was washed with DMF (6×) and capped with 1 M acetic anhydride (Ac₂O) and 1 M Et₃N in 150 mL DMF for 45 min at 25° C., followed by washing with DMF (4×) and CH₂Cl₂ (3×).

A small portion of the resin was subjected to analysis for Fmoc group. The resin (3 samples, 10-20 mg each sample) was weighed into three scintillation vials. Fmoc group removal was achieved using 0.5 mL piperidine:DMF (1:1 (v/v)). The solution was added to the resin samples, placed on a platform shaker and allowed to shake gently for 1 hr. Then, the samples were removed, diluted with 20 mL of HPLC grade methanol, and mixed thoroughly. After allowing resin to settle, 1 mL of the resulting solution was again diluted to 10 mL using HPLC grade methanol. The samples were subjected to UV-Visible spectrometry at 301 nm indicating a substitution level of 0.15 mmol/g.

The resin was washed with DMF (3×) and treated with piperidine:DMF (1:4 (v/v), 2×10 min) to achieve Fmoc group removal and washed again with DMF (8×). N^{α},N^{ε}-Bis(5-thio-2-nitrobenzoyl)-L-lysine disulfide (DTNB-Lys-OH) (2.3 g, 4.5 mmol), BOP (2.0 g, 4.5 mmol), and HOBt (0.70 g, 4.5 mmol) were combined and dissolved in 100 mL DMF, and then NMM (0.83 ml, 4.5 mmol) was added. The solution was added to the resin and shaken for 12 h. After washing with DMF (6×) and CH₂Cl₂ (3×), completion of acylation was confirmed by a ninhydrin test. The resin was then washed with ethyl ether (Et₂O) (3×), CH₂Cl₂ (3×), Et₂O (3×), and then dried in vacuo. The yield was 22.48 grams.

### Selection of Model Peptides Each Containing an Intramolecular Disulfide

The model peptides selected as synthetic targets for oxidation are shown in Table II. These include Arg⁸-Vasopressin: 1: 9 residues, disulfide bridge between residues 1 and 6), an erythropoietin mimic; 2: 14 residues, disulfide bridge between residues 3 and 12), urotensin II (U II); 3; 11 residues, disulfide bridge between residues 5 and 10), a purely synthetic construct; 4: 7 residues, disulfide bridge between residues 1 and 6), a U II potent agonist; 5: 8 residues, disulfide bridge between residues 2 and 7) and a U II potent antagonist; and 6: 8 residues, disulfide bridge between residues 2 and 7).

The first three examples represent common, naturally occurring peptides or their analogues; in particular, urotensin II (3) is the most potent mammalian peptide vasoconstrictor known to date. Peptide 4 is a purely artificial construction without any known biological action, designed to represent a medium-sized disulfide-containing cyclic peptide that incorporates two of the most troublesome residues (Trp and Met) that are prone to side reactions when carrying out solution-based oxidations. Newly reported urotensin II agonist, H-Asp-cyclo[Pen-Phe-Trp-Lys-Tyr-Cys]-Val-OH 5 (Grieco, et. al., A new, potent urotensin II receptor peptide agonist containing a pen residue at the disulfide bridge, J. Med. Chem. 45 4391-4394 (2002), as well as a U II antagonist 6 (Patacchini Et. al, Urantide: an ultrapotent urotensin II antagonist peptide in the rat aorta, Br. J. Pharm. 140, 1155-1158 (2003)), were chosen as challenging test sequences, due to the presence in each of a penicillamine residue, which represents a highly constrained, sterically hindered cysteine replacement.

### Small Scale Oxidation Procedure Using Clear-OX™ Resin

Prior to use, CLEAR-OX™ resin was allowed to swell for 30 min in CH₂Cl₂ and then washed with DMF, MeOH, and CH₃CN:H₂O (1:1 v/v). The reduced peptides (peptides 1 through 6, 20 mg each) were dissolved in degassed 0.1 M ammonium acetate buffer/acetonitrile (1:1 v/v) at 6-7 mg/mL concentration levels. Each peptide solution was added to pre-swollen CLEAR-OX™ resin (0.2 meq/g; 3-fold molar excess over the amount of peptide, ˜200-400 mg of CLEAR-OX™), and the reaction mixture was shaken at 25° C. for 2 h. Progress of the oxidation was noted as the color of the resin changed from yellow to deep orange. Reaction completion was confirmed by Ellman's test (Ellman, G. L. (1959) Tissue sulfydryl groups. Arch. Biochem. Biophys. 82, 70-77). The reaction mixture was filtered and washed with a small amount of CH₃CN:H₂O (1:1 v/v). The filtrates were concentrated in vacuo to remove CH₃CN, lyophilized to form powders, and then analyzed by RP-HPLC and ES-MS as set forth in Table 2 which shows the mass spectral analyses and chromatography data for disulfide-bridged peptides obtained by CLEAR-OX™-mediated oxidation compared to solution-phase oxidation methods.

### Solution Oxidation

The reduced peptides (20 mg) were dissolved in 40 mL of degassed 0.1% aqueous acetic acid, and the pH was adjusted to 7.5-8.0 with 8 M aqueous ammonium hydroxide. Each solution was titrated at 25° C. with 0.01 M K₃Fe(CN)₆ until the yellow color was maintained for 10 min. Reaction completeness was confirmed by Ellman's test (Ellman, G. L. (1959) Tissue sulfhydryl groups. Arch. Biochem. Biophys. 82, 70-77). The pH of the solution was lowered to 6-7, ion-exchange resin (0.5 g of AG3×4, acetate form) was added, and stirring was continued for 30 min. The suspension was then filtered to remove resin, and the resin was washed further with additional small amounts of water (2×5 mL). The combined filtrates were lyophilized, the residue was resuspended in water, and lyophilized for two additional cycles. The obtained products were analyzed by RP-HPLC and ES-MS as set forth in Table II below.

### Preparative Oxidation of Urotensin II Agonist Using Clear-OX™ Resin

Prior to use, CLEAR-OX™ resin was allowed to swell for 30 min in CH₂Cl₂, and then washed with DMF, MeOH, and CH₃CN:H₂O (1:1 v/v). The reduced urotensin II peptide agonist, H-Asp-Pen-Phe-Trp-Lys-Tyr-Cys-Val-OH (1.5 g), was dissolved in degassed AcOH:H₂O (1:1 v/v) (10 mL) plus CH₃CN:H₂O (1:1 v/v) (3 mL). The solution was diluted with degassed CH₃CN:H₂O (1:1 v/v) (300 mL), and the pH of the solution was adjusted to ˜4 with 8 M aqueous ammonium hydroxide. CLEAR-OX™ resin (13.74 g) slurry in degassed CH₃CN:H₂O (1:1 v/v) was added to the peptide solution, and the mixture was shaken for 2 h at 25 C. Progress of the oxidation was noted as the color of the resin changed from yellow to deep orange. Reaction completion was confirmed by an Ellman's test (23). The resin-bound oxidant was removed by filtration. The resin was washed with CH₃CN:H₂O (1:1 v/v) (7×60 mL), and filtrates were concentrated in vacuo to remove CH₃CN, and then lyophilized. Lyophilized material was dissolved in water (100 mL) and lyophilized again to remove volatile salts. The process was repeated two more times and the product obtained (0.64 g, 42%) was analyzed by RP-HPLC and ES-MS. Further preparative purification using Vydac C18 column (50×300 mm) gave 81 mg of final product. Amino acid analysis: Asp 1.03 (1), Tyr 0.61 (1), Val 1.01 (1), Lys 1.03 (1), Phe 0.94 (1), Cys and Trp not determined. ES-MS: calc 1088.45 found 1089.45 [M+H]+.

### Preparative Oxidation of Urotensin II Antagonist Using Clear-OX™ Resin

The reduced urotensin antagonist, H-Asp-Pen-Phe-D-Trp-Om-Tyr-Cys-Val-OH, (2.0 g) was oxidized as described for the agonist. Crude oxidized product (1.56 g, 78.3%) was further purified using Vydac C18 column (50×300 mm). The main fractions were pooled and lyophilized to yield 383 mg of homogenous product. ES-MS: calc 1074.43 found 1075.50[M+H]+.

### Oxidation Results

First, the linear, reduced peptides were assembled according to standard Fmoc/tBu solid-phase synthesis strategies, and cleaved from the supports using appropriate TFA/scavenger cocktails. The crude peptides were then used directly, without further purification, in oxidation studies. Solutions of reduced peptides in degassed 0.1 M ammonium acetate buffer/acetonitrile (1:1 v/v), at 6-7 mg/mL concentration levels, were added to CLEAR-OX™ resin slurry. Cyclic products were isolated by simple filtration, and then analyzed to determine crude purities and yields. Solution-phase oxidations were achieved using standard protocols involving excess K₃Fe(CN)₆ as the oxidant (Andreu, D., Albericio, F., Solé, N. A., Munson, M. C., Ferrer, M. & Barany, G. (1994) Formation of disulfide bonds in synthetic peptides and proteins. In Methods in Molecular Biology, Vol. 35: Peptide Synthesis Protocols (Pennington, M. W. & Dunn, B. M., eds.) Humana Press, Totowa, N.J., pp. 91-169; Hope, D. B., Murti, V. V. S. & du Vigneaud, V. (1962) A highly potent analogue of oxytocin, desamino-oxytocin. J. Biol. Chem. 237, 1563-1566.) at pH levels of 7.5-8.0 with peptide concentrations of 0.5 mg/mL. Once an endpoint was reached, excess inorganic reagents and by-products were removed by added ion-exchange resin.

Results of oxidation studies are presented in the following Table II as follows:

TABLE II
Mass spectral analyses and chromatography data for disulfide-bridged peptides obtained by CLEAR-OX ™-
mediated compared to solution-phase oxidation methods

| | Mass Spectral Analysis | | | | HPLC Purity* (%) | | |
|---|---|---|---|---|---|---|---|
| (No.) | MW | MW [M + H]⁺ | MW | MW [M + H]⁺ | CLEAR- | CLEAR- | Solution |
| Peptide | Reduced | Reduced | Oxidized | Oxidized | OX Resin | OX Resin | Oxidation |
| Sequence | Theory | Found | Theory | Found | pH = 4.6 | pH = 6.8 | pH = 7.5-8 |
| 1 Arg⁸-Vasopressin | 1085.45 | 1086.48 | 1083.44 | 1084.46 | 57 | 51 | 28 |
| (AVP) | | | | | | | |
| H-c[Cys-Tyr-Phe- | | | | | | | |
| Gln-Asn-Cys]-Pro- | | | | | | | |
| Arg-Gly-NH₂ | | | | | | | |
| 2 Erythropoietin | 1373.67 | 1374.72 | 1371.65 | 1372.68 | 26 | 28 | 32 |
| Mimic | | | | | | | |
| H-Gly-Gly-c[Cys- | | | | | | | |
| Arg-Ile-Gly-Pro-Ile- | | | | | | | |
| Thr-Trp-Val-Cys]- | | | | | | | |
| Gly-Gly-NH₂ | | | | | | | |
| 3 Urotensin II | 1389.57 | 1390.60 | 1387.56 | 1388.59 | 54 | 44 | 28 |
| H-Glu-Thr-Pro-Asp- | | | | | | | |
| c[Cys-Phe-Trp-Lys- | | | | | | | |
| Tyr-Cys]-Val-OH | | | | | | | |
| 4 Met/Trp-Containing | 810.33 | 811.34 | 808.32 | 809.34 | 50 | 43 | 42 |
| Model | | | | | | | |
| H-c[Cys-Trp-Ala- | | | | | | | |
| Met-Ala-Cys]-Lys- | | | | | | | |
| NH₂ | | | | | | | |
| 5 Urotensin II Potent | 1090.46 | 1091.48 | 1088.45 | 1089.47 | 42 | 38 | 21 |
| Agonist | | | | | | | |
| H-Asp-c[Pen-Phe- | | | | | | | |
| Trp-Lys-Tyr-Cys]- | | | | | | | |
| Val-OH | | | | | | | |
| 6 Urotensin II | 1076.46 | 1077.49 | 1074.43 | 1075.28 | 36 | 35 | 19 |
| Antagonist | | | | | | | |
| H-Asp-c[Pen-Phe-D- | | | | | | | |
| Trp-Orn-Tyr-Cys]- | | | | | | | |
| Val-OH | | | | | | | |

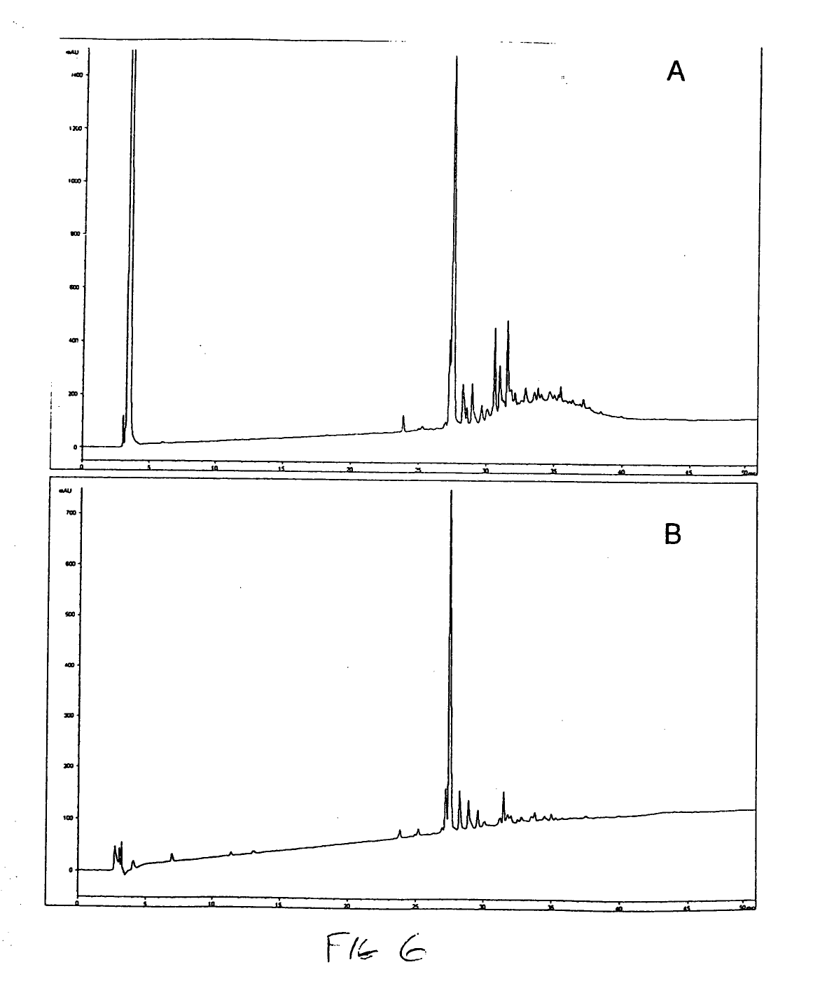

*The value given expresses in % the area of the major peak, relative to the total area of all peaks in the HPLC chromatogram. See FIG. 6 for representative chromatogram.

In the majority of tested examples, oxidations mediated by CLEAR-OX™ resulted in the expected products with good yields (40-90%, crude peptide). Purities of the crude cyclic oxidized products obtained by the CLEAR-OX™ method were generally higher than those obtained in the corresponding solution oxidation controls. Initial CLEAR-OX™ experiments were performed at various excess ratios, and it was shown that ratios as low as 2 equivalents of CLEAR-OX™ to reduced peptide were sufficient to achieve effective oxidations. Whereas traditional oxidation methods require high dilution to minimize dimer formation, concentration of the peptide is much less of a factor due to the pseudo-dilution effect of the CLEAR-OX™ resin. Thus, oxidations using CLEAR-OX™ were carried out at much higher concentrations than solution oxidations (6-7 vs 0.5 mg/mL), meaning that far less solvent would be required for larger scale reactions.

In general, oxidations with CLEAR-OX™ were completed within 1-2 h, even in the cases where the sequence included a sterically-hindered penicillamine residue (5 and 6). CLEAR-OX™ was found to be compatible with wide ranges of pH used in typical oxidation reactions, i.e., pH 2 to 8. Solubility problems were overcome by the addition of acetonitrile to CLEAR-OX™-mediated cyclization mixtures; our studies suggest that acetonitrile:aqueous (CH₃CN:H₂O) mixtures serve well as a general milieu for oxidations. This solvent combination has been proven effective at solubilizing the majority of the synthetic peptides, and is fully compatible with CLEAR-OX™ resin. Moreover, reactants may be separated from the polymer-bound oxidant by simple filtration, hence circumventing an often troublesome step in solution-phase techniques. Reactions performed at medium scale for difficult sequences 5 and 6 (1 to 2 grams of reduced peptide) demonstrated the effectiveness and convenience of CLEAR-OX™ for the preparation of disulfide-bridged peptides under mild conditions. Most significant, for these difficult oxidations of penicillamine-containing sequences, the use of CLEAR-OX™ proved superior over solution methods in terms of yield and purity as shown in FIG. 6.

All percentages and ratios used herein are weight percentages and ratios unless otherwise indicated. All publications, patents and patent documents cited are fully incorporated by reference herein, as though individually incorporated by reference. Numerous characteristics and advantages of the invention meant to be described by this document have been set forth in the foregoing description. It is to be understood, however, that while particular forms or embodiments of the invention have been illustrated, various modifications, including modifications to shape, and arrangement of parts, and the like, can be made without departing from the spirit and scope of the invention.

1. A reagent for preparation of disulfide-bridged peptides, said reagent comprising an oxidative functionality bound to a cross-linked ethoxylate acrylate resin polymer and having the formula: 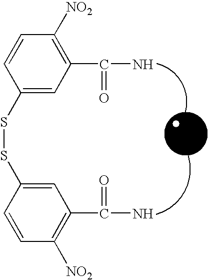
wherein \textcircled{R} is a cross-linked ethoxylate acrylate resin polymer prepared by reacting an olefin-containing monomer and a multifunctional (meth)acrylate crosslinker, wherein the multifunctional (meth)acrylate crosslinker has the following formula: 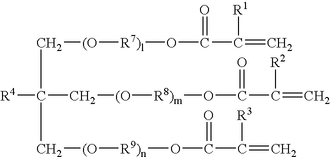
wherein:
(i) R¹, R², and R³ are each independently hydrogen or a methyl group,
(ii) R⁴ is hydrogen or an organic group or substituent that can interact in the polymerization and/or crosslinking process or is nonreactive under the conditions of the polymerization and/or crosslinking process,
(iii) R⁷, R⁸, and R⁹ are each independently —CH₂—CH₂—, —CH₂—CH₂—CH₂—, —CH₂—CH(CH₃)—, or —CH(CH₃)—CH₂—, and
(iv) each of l, m, and n is no greater than about 100 with the proviso that at least one of l, m, or n is at least 1.

2. The reagent of claim 1, wherein the sum of l+m+n is about 14.

3. The reagent of claim 1, wherein the oxidative functionality is bound to the cross-linked ethoxylate acrylate resin polymer via a spacer moiety.

4. The reagent of claim 3, wherein the spacer moiety is a linking group comprising one or more amino acid residues.

5. The reagent of claim 1, said reagent having the formula: 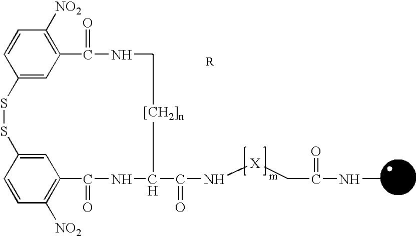
wherein n=1-8,
X=(CH₂) or (CH₂CH₂O)
and m=0-12.

6. The reagent of claim 5, wherein n=4.

7. The reagent of claim 1, said reagent having the formula: 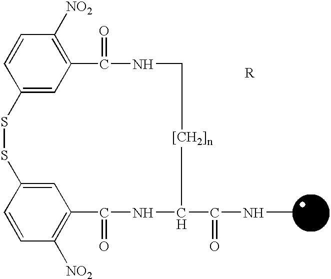
wherein n=1-8.

8. The reagent of claim 7, wherein n=4.

9. A method for preparing disulfide-bridged peptides comprising contacting a peptide solution comprising one or more peptides having two or more thiol functionalities with the reagent of claim 1 under conditions suitable for oxidation of the thiol functionalities to form peptides having intramolecular peptide disulfide bonds.

10. The method of claim 9, wherein the peptide solution comprises a peptide having two or more thiol functionalities, and the peptide solution is contacted with the reagent under conditions suitable for oxidation of the thiol functionalities to form peptides having intramolecular peptide disulfide bonds.

11. The method of claim 9, wherein the peptide solution comprises two or more polythiol peptides as a peptide mixture, and the peptide solution is contacted with the reagent under conditions suitable for oxidation of the thiol functionalities to form a corresponding mixture of peptides having intramolecular peptide disulfide bonds.

12. The method of claim 9, wherein the peptide solution has peptide a concentration of from about 4 mg/ml to about 7 mg/ml.

13. The method of claim 9, wherein the ratio of excess reagent to reduced peptide is from about 2 to about 5.

14. The method of claim 9, wherein the peptide solution comprises an acetonitrile/aqueous mixed solvent system.

15. A method of making the reagent of claim 1, comprising:
a) providing a cross-linked ethoxylate acrylate resin polymer by reacting an olefin-containing monomer and a multifunctional (meth)acrylate crosslinker, wherein the multifunctional (meth)acrylate crosslinker has the following formula: 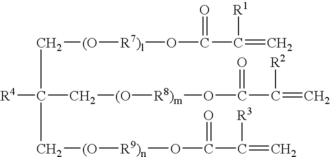
wherein:
(i) R¹, R², and R³ are each independently hydrogen or a methyl group,
(ii) R⁴ is hydrogen or an organic group or substituent that can interact in the polymerization and/or crosslinking process or is nonreactive under the conditions of the polymerization and/or crosslinking process,
(iii) R⁷, R⁸, and R⁹ are each independently —CH₂—CH₂—, —CH₂—CH₂—CH₂—, —CH₂—CH(CH₃)—, or —CH(CH₃)—CH₂—, and
(iv) each of l, m, and n is no greater than about 100 with the proviso that at least one of l, m, or n is at least 1;
b) binding a bifunctional amino acid anchor to the cross-linked ethoxylate acrylate resin polymer;
c) attaching two 2-nitro-5-thiobenzoic acid compounds wherein the sulfur is protected by a sulfur protecting group to the resin-bound bifunctional amino acid anchor to form two sulfur protected 2-nitro-5-thiobenzoic acid residues;
d) removing the sulfur protecting groups; and
e) oxidizing the two 2-nitro-5-thiobenzoic acid residues to form 5,5′-dithiobis(2-nitrobenzoic acid residues bound to the to the cross-linked ethoxylate acrylate resin polymer.

16. The method of claim 15, wherein the bifunctional amino acid anchor comprises a lysine residue.

17. A method of making the reagent of claim 1, comprising:
a) providing a cross-linked ethoxylate acrylate resin polymer by reacting an olefin-containing monomer and a multifunctional (meth)acrylate crosslinker, wherein the multifunctional (meth)acrylate crosslinker has the following formula: 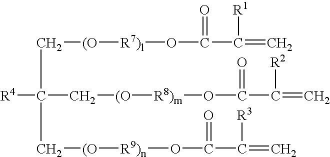
wherein:
(i) R¹, R², and R³ are each independently hydrogen or a methyl group,
(ii) R⁴ is hydrogen or an organic group or substituent that can interact in the polymerization and/or crosslinking process or is nonreactive under the conditions of the polymerization and/or crosslinking process,
(iii) R⁷, R⁸, and R⁹ are each independently —CH₂—CH₂—, —CH₂—CH₂—CH₂—, —CH₂—CH(CH₃)—, or —CH(CH₃)—CH₂—, and
(iv) each of l, m, and n is no greater than about 100 with the proviso that at least one of l, m, or n is at least 1;
b) binding a bifunctional amino acid anchor in solution with 5,5′-dithiobis(2-nitrobenzoic acid) (“DTNB”) to form a DTNB-bifunctional amino acid anchor derivative; and
c) binding the DTNB-bifunctional amino acid anchor derivative to the cross-linked ethoxylate acrylate resin polymer.

18. The method of claim 17, wherein the bifunctional amino acid anchor comprises a lysine residue.

19. The method of claim 17, wherein the DTNB-bifunctional amino acid anchor derivative is bound directly to the cross-linked ethoxylate acrylate resin polymer.

20. The method of claim 17, wherein the DTNB-bifunctional amino acid anchor derivative is bound to the cross-linked ethoxylate acrylate resin polymer via a spacer moiety.
